(19) United States
(12) Patent Application Publication
(10) Pub. No.: US 2007/0160689 A1
(43) Pub. Date: Jul. 12, 2007

(54) Compositions and methods for treatment of coughing, sneezing, rhinorrhea, and/or nasal obstruction

(75) Inventors: John A. Giordano, West Orange, NJ (US); Kevin M. Brown, Harrington Park, NJ (US)

Correspondence Address: Kirkpatrick & Lockhart Preston Gates Ellis LLP, 1735 NEW YORK AVENUE, NW, SUITE 500, WASHINGTON, DC, 20006, US

(73) Assignee: Everett Laboratories, Inc.

(21) Appl. No.: 11/330,351

(22) Filed: Jan. 12, 2006

(51) Int. Cl. A61K 36/49; A61K 31/485; A61K 31/185; A61K 31/205; A61K 31/137

(52) U.S. Cl. 424/725.1; 514/554; 514/282; 514/649; 514/553; 514/288

(57) ABSTRACT

The present invention relates to compositions that comprise an expectorant, an extended release antitussive, and an extended release decongestant. Specifically, the compositions comprise guaifenesin, phenylephrine tannate, and dextromethorphan tannate. The present invention also includes methods for using these compositions for treatment of patients suffering from, for example and without limitation, coughing, sneezing, rhinorrhea, and/or nasal obstruction.

### FIELD OF THE INVENTION

The present invention relates to compositions that comprise an expectorant, an extended release antitussive, and an extended release decongestant. Specifically, the compositions comprise guaifenesin, phenylephrine tannate, and dextromethorphan tannate. The present invention also includes methods for using these compositions for treatment of patients suffering from, for example and without limitation, coughing, sneezing, rhinorrhea, and/or nasal obstruction.

### BACKGROUND OF THE INVENTION

People suffering from coughing, sneezing, rhinorrhea, and/or nasal obstruction commonly take throat lozenges, cough syrups or cough drops for symptomatic relief. While many such medications presently exist, there is room for improvement in the composition of these medications. Many medications contain a combination or variety of antitussives, expectorants and/or decongestants. While a combination or variety may be acceptable to some patients, others may have restrictions due to allergies or other incompatibilities with certain ingredients. Further, many of these medications contain sugar and alcohol while customers would prefer a medication that did not include these substances. Therefore, there is a need for a coughing, sneezing, rhinorrhea, and/or nasal obstruction medication that is free of added sugar and added alcohol that is also restricted to the inclusion of specific antitussives, expectorants, and decongestants.

Guaifenesin is an expectorant which increases the output of phlegm (sputum) and bronchial secretions by reducing adhesiveness and surface tension. The increased flow of less viscid secretions promotes ciliary action and changes a dry, unproductive cough to one that is more productive and less frequent.

Phenylephrine is a sympathomimetic nasal decongestant which acts predominately on alpha adrenergic receptors in the mucosa of the respiratory tract, producing vasoconstriction with minimal action on beta receptors. It functions as an oral nasal decongestant with minimal central nervous system (CNS) stimulation and promotes sinus drainage.

Dextromethorphan acts as an antitussive in suppressing the cough reflex in the medulla. Treatment is intended to relieve cough frequency without abolishing protective cough reflex.

Tannate salts of active agents are known to provide therapeutic effect for longer periods of time. It is believed that the inclusion of an active agent in a tannate salt form extends the release profile of the active agent and there is less spiking in pharmacological effect of the active agent. The active agent can therefore be administered to the patient less often and there are fewer side effects, particularly from over dosage effects. See, U.S. Pat. Nos. 5,599,846, and 5,663,415, issued Feb. 4, 1997 and Sep. 2, 1997, respectively, for further information on the preparation and use of phenylephrine tannate. See, U.S. Pat. Nos. 6,670,370 and 6,509,492, issued Dec. 30, 2003 and Jan. 21, 2003, respectively, for further information on the preparation and use of dextromethorphan tannate.

### SUMMARY OF THE INVENTION

The present invention provides compositions and methods of using these compositions for the therapeutic treatment of coughing, sneezing, rhinorrhea, and/or nasal obstruction. Specifically, for example, the present invention relates to novel compositions of antitussives, expectorants, and decongestants that can be used to treat coughing, sneezing, rhinorrhea, and/or nasal obstruction caused by a variety of factors.

In one embodiment of the present invention, the compositions may comprise dextromethorphan tannate, phenylephrine tannate and guaifenesin and may be administrable to a patient.

In another embodiment of the present invention, the compositions may be substantially free of other added active ingredients. For example, in one embodiment, the compositions of the present invention may be substantially free of another added antitussive. Specifically, the compositions may be substantially free of, for example and without limitation, one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan hydrobromide, chlophendianol hydrochloride, carbetapentane citrate, caramiphen edisylate, noscapine, noscapine hydrochloride, and menthol.

In another embodiment of the present invention, the compositions may be substantially free of other added active ingredients, such as, for example and without limitation, another decongestant. In one embodiment of the present invention, the compositions may be substantially free of any other added nasal decongestant. In another embodiment, the compositions of the present invention may be substantially free of any other added decongestants such as, for example and without limitation, one or more of the group consisting of ephedrine, ephedrine sulfate, ephedrine hydrochloride, pseudoephedrine hydrochloride, phenylephrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

In another embodiment of the present invention, the compositions may be substantially free of other added active ingredients, such as, for example and without limitation, another opioid analgesic. In one embodiment the compositions of the present invention may be substantially free of other added opioid analgesics such as, for example and without limitation, one or more of the group consisting of codeine, morphine, hydromorphone, hydrocodone, oxymorphone, levorphanol, fentanyl, propoxyphene, diphenoxylate, meperidine, methadone, oxycodone, butorphanol, benzonate and morphine.

In another embodiment of the present invention, the compositions may be substantially free of other added active ingredients, such as, for example and without limitation, another expectorant. In one embodiment the compositions of the present invention may be substantially free of other added expectorants such as, for example and without limitation, one or more of the group consisting of ammonium chloride, ammonium carbonate, acetylcysteine, antimony potassium tartrate, glycerin, potassium iodide, sodium citrate, terpin hydrate, and tolu balsam.

In another embodiment of the present invention, the compositions may be substantially free of added sugar. In another embodiment, the compositions of the present invention may be substantially free of added alcohol. In another embodiment of the present invention, the compositions may be substantially free of added sugar and substantially free of added alcohol.

In another embodiment of the present invention, the compositions may be in a liquid form. In another embodiment of the present invention, the compositions may further comprise a flavorant such as, for example and without limitation, a natural flavorant or an artificial flavorant. Flavorants included in the compositions of the present invention may include, for example and without limitation, anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Flavorants included in the compositions of the present invention also may include fruit essence, for example and without limitation, apple essence, pear essence, peach essence, berry essence, wildberry essence, date essence, blueberry essence, kiwi essence, strawberry essence, raspberry essence, cherry essence, plum essence, pineapple essence, and apricot essence. Further, flavorants included in the compositions of the present invention also may include, for example and without limitation, natural mixed berry flavor, citric acid, malic acid, vanilla, vanillin, cocoa, chocolate, and menthol.

In a specific embodiment, the compositions of the present invention may also include a non-sugar sweetening agent. For example, the compositions of the present invention may further comprise, without limitation, saccharin sodium, maltodextrin, aspartame, potassium acesulfame, neohesperidin dihydrochalcone, sucralose, monoammonium glycyrrhizinate, and mixtures thereof.

In one embodiment of the present invention, the composition may further comprise citric acid, adetate disodium, glycerin, methylparaben, propylparaben, propylene glycol, saccharin sodium, sodium citrate, sorbitol, water, FD&C red 40, and artificial cherry flavor.

In another embodiment of the present invention, the compositions may comprise about 28 mg/5 ml to about 48 mg/5 ml dextromethorphan tannate; about 14 mg/5 ml to about 24 mg/5 ml phenylephrine tannate; and/or about 75 mg/5 ml to about 125 mg/5 ml guaifenesin.

In another embodiment of the present invention, the compositions may comprise about 34 mg/5 ml to about 42 mg/5 ml dextromethorphan tannate; about 17 mg/5 ml to about 21 mg/5 ml phenylephrine tannate; and/or about 90 mg/5 ml to about 110 mg/5 ml guaifenesin.

In another embodiment of the present invention, the compositions may comprise about 36 mg/5 ml to about 40 mg/5 ml dextromethorphan tannate; about 18 mg/5 ml to about 20 mg/5 ml phenylephrine tannate; and/or about 95 mg/5 ml to about 105 mg/5 ml guaifenesin.

In another embodiment of the present invention, the compositions may comprise about 38 mg/5 ml dextromethorphan tannate; about 19 mg/5 ml phenylephrine tannate; and/or about 100 mg/5 ml guaifenesin.

The present invention also includes methods of administering the compositions of the invention to patients. In some embodiments, the composition of the invention may be administered to the patient orally. In some embodiments, the composition may be administered to the patient at a frequency of once a day, twice a day, three times a day or four times a day. In some embodiments, the composition may be administered to the patient in a dose from about 0.1 ml to about 100 ml.

In some embodiments, the patient may be suffering from coughing, sneezing, rhinorrhea, nasal obstruction, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, a respiratory disease, a cold, acute bronchitis, chronic bronchitis, asthmatic bronchitis, bronchiectasis, pneumonia, lung tuberculosis, silicosis, silicotuberculosis, pulmonary cancer, upper respiratory inflammation, pharyngitis, laryngitis, nasal catarrh, asthma, bronchial asthma, infantile asthma, pulmonary emphysema, pneumoconiosis, pulmonary fibrosis, pulmonary silicosis, pulmonary suppuration, pleuritis, tonsillitis, cough hives, and/or whooping cough.

In one embodiment of the present invention, the methods may utilize compositions comprising dextromethorphan tannate, phenylephrine tannate and guaifenesin.

In another embodiment of the present invention, the methods may utilize compositions that may be substantially free of other added active ingredients. For example, in one embodiment, the methods may utilize compositions that may be substantially free of another added antitussive. Specifically, the methods may utilize compositions that may be substantially free of, for example and without limitation, one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan hydrobromide, chlophendianol hydrochloride, carbetapentane citrate, caramiphen edisylate, noscapine, noscapine hydrochloride, and menthol.

In another embodiment of the present invention, the methods may utilize compositions that may be substantially free of other added active ingredients, such as, for example and without limitation, another decongestant. In one embodiment of the present invention, the methods may utilize compositions that may be substantially free of any other added nasal decongestant. In another embodiment, the methods may utilize compositions that may be substantially free of any other added decongestants such as, for example and without limitation, one or more of the group consisting of ephedrine, ephedrine sulfate, ephedrine hydrochloride, pseudoephedrine hydrochloride, phenylephrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

In another embodiment of the present invention, the methods may utilize compositions that may be substantially free of other added active ingredients, such as, for example and without limitation, another opioid analgesics. In one embodiment, the methods may utilize compositions that may be substantially free of other added opioid analgesics such as, for example and without limitation, one or more of the group consisting of codeine, morphine, hydromorphone, hydrocodone, oxymorphone, levorphanol, fentanyl, propoxyphene, diphenoxylate, meperidine, methadone, oxycodone, butorphanol, benzonate and morphine.

In another embodiment of the present invention, the methods may utilize compositions that may be substantially free of other added active ingredients, such as, for example and without limitation, another expectorant. In one embodiment, the methods may utilize compositions that may be substantially free of other added expectorants such as, for example and without limitation, one or more of the group consisting of ammonium chloride, ammonium carbonate, acetylcysteine, antimony potassium tartrate, glycerin, potassium iodide, sodium citrate, terpin hydrate, and tolu balsam.

In another embodiment of the present invention, the methods may utilize compositions that may be substantially free of added sugar. In another embodiment, the methods may utilize compositions that may be substantially free of added alcohol. In another embodiment of the present invention, the methods may utilize compositions that may be substantially free of added sugar and substantially free of added alcohol. In another embodiment of the present invention, the methods may utilize compositions that may be in a liquid form. In another embodiment of the present invention, the methods may utilize compositions that may include a flavorant such as, for example and without limitation, a natural flavorant or an artificial flavorant. Flavorants included in the compositions of the present invention may include, for example and without limitation, anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Flavorants included in the compositions of the present invention also may include fruit essence, for example and without limitation, apple essence, pear essence, peach essence, berry essence, wildberry essence, date essence, blueberry essence, kiwi essence, strawberry essence, raspberry essence, cherry essence, plum essence, pineapple essence, and apricot essence. Further, flavorants included in the compositions of the present invention also may include, for example and without limitation, natural mixed berry flavor, citric acid, malic acid, vanilla, vanillin, cocoa, chocolate, and menthol.

In a specific embodiment, the compositions of the present invention may also include a non-sugar sweetening agent. For example, the methods may utilize compositions that may include, without limitation, saccharin sodium, maltodextrin, aspartame, potassium acesulfame, neohesperidin dihydrochalcone, sucralose, monoammonium glycyrrhizinate, and mixtures thereof.

In one embodiment of the present invention, the methods may utilize compositions that may include citric acid, adetate disodium, glycerin, methylparaben, propylparaben, propylene glycol, saccharin sodium, sodium citrate, sorbitol, water, FD&C red 40, and artificial cherry flavor.

In another embodiment of the present invention, the methods may utilize compositions that may comprise about 28 mg/5 ml to about 48 mg/5 ml dextromethorphan tannate; about 14 mg/5 ml to about 24 mg/5 ml phenylephrine tannate; and/or about 75 mg/5 ml to about 125 mg/5 ml guaifenesin.

In another embodiment of the present invention, the methods may utilize compositions that may comprise about 34 mg/5 ml to about 42 mg/5 ml dextromethorphan tannate; about 17 mg/5 ml to about 21 mg/5 ml phenylephrine tannate; and/or about 90 mg/5 ml to about 110 mg/5 ml guaifenesin.

In another embodiment of the present invention, the methods may utilize compositions that may comprise about 36 mg/5 ml to about 40 mg/5 ml dextromethorphan tannate; about 18 mg/5 ml to about 20 mg/5 ml phenylephrine tannate; and/or about 95 mg/5 ml to about 105 mg/5 ml guaifenesin.

In another embodiment of the present invention, the methods may utilize compositions that may comprise about 38 mg/5 ml dextromethorphan tannate; about 19 mg/5 ml phenylephrine tannate; and/or about 100 mg/5 ml guaifenesin.

Other objectives, features and advantages of the present invention will become apparent from the following detailed description. The detailed description and the specific examples, although indicating specific embodiments of the invention, are provided by way of illustration only. Accordingly, the present invention also includes those various changes and modifications within the spirit and scope of the invention that may become apparent to those skilled in the art from this detailed description.

### DETAILED DESCRIPTION OF THE INVENTION

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a decongestant” is a reference to one or more decongestants and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “patient,” as used herein, comprises any and all organisms and includes the term “subject.” “Patient” may refer to a human or any other animal.

The term “administrable” defines a composition that is able to be given to a patient. Likewise, “administering” refers to the act of giving a composition to a patient or otherwise making such composition available to a patient.

The term “active ingredient” as used herein is any ingredient that is an antitussive, a decongestant or an expectorant when taken orally by a patient.

The term “substantially free,” as used herein, means free from therapeutically effective amounts of compounds when administered in suggested dosages, but may include trace amounts of compounds in non-therapeutically effective amounts.

Through the inclusion of an antitussive, a decongestant and an expectorant, the compositions and methods of the present invention may alleviate symptoms, such as coughing, sneezing, rhinorrhea, and/or nasal obstruction caused by a variety of factors.

Antitussive drugs may act peripherally to inhibit cough by suppressing the responsiveness of one or more vagal sensory receptors that produce cough. Bolser et al., 86(3) J. APPL. PHYSIOL. 1017-1024 (1999); Adcock, RESPIR. MED. 85, Suppl. A 43-46 (1991); Bolser, 9 PULM. PHARMACOL. 357-364 (1997); Kase, I TRENDS PHARMACOL. Sci. 237-239 (1980). Antitussive drugs also may act within the central nervous system at the level of the brain stem, where the basic neural circuitry responsible for cough is located. Bolser et al., supra; Korpas & Tomori, COUGH AND OTHER RESPIRATORY REFLEXES, New York, Karger (1979), Shannon, 9 PULM. PHARWACOL. 343-347 (1997); Shannon et al., NEURAL CONTROL OF BREATHING, edited by Miller et al. 215-224 (1996). Specifically, centrally-acting antitussives are thought to inhibit cough by interfering with the central modulation of afferent signals from the periphery, thereby decreasing sensitivity of the cough center located within the medulla to incoming stimuli. Even more specifically, a recent model of the basic cough circuitry suggests that the eupneic respiratory pattern and the cough motor pattern are produced by essentially the same neural components. Although this pattern generator normally controls breathing, its behavior is modified to produce cough by excitatory inputs from medullary second-order interneurons mediating pulmonary rapidly and slowly adapting receptor (RAR and SAR, respectively)-afferent information. Shannon, supra; Shannon et al., supra. Centrally active antitussive drugs may act at any level within this system. For example, these drugs could suppress the responsiveness of components of the central pathway for transmitting vagal sensory information (second-order interneurons) and/or have more complex effects on the motor pattern generator for cough. Bolser & DeGennaro, 662 BRAIN RES. 25-30 (1994); Bolser et al., 113 BR. J. PHARMACOL. 1344-1348 (1994); Chou & Wang, 223 J. PHARMACOL. EXP. THER. 249-253 (1975).

Dextromethorphan (DXM) is a potent antitussive. Dextromethorphan (d-3-methoxy-N-methyl-morphinan) is the dextro isomer of levomethorphan. It has for some time been used as a cough suppressant and may be useful for pain treatment and protection against nerve cell damage. The drug acts centrally, in a manner independent of that of opioids (e.g., codeine), to elevate the threshold for coughing by an, as yet, ill-defined mechanism. DXM, as well as its metabolite, methorphan, have been shown to be antagonists at the N-methyl-d-aspartate (NMDA) receptor which is linked to other amino acid neurotransmitters, glutamate and glycine. In one specific embodiment, dextromethorphan tannate may be used. In another specific embodiment of the compositions and methods of the present invention, dextromethorphan tannate may be included in amounts ranging from about 36 mg/5 ml to about 44 mg/5 ml. In another specific embodiment of the compositions and methods of the present invention, dextromethorphan tannate may be included in amounts ranging from about 34 mg/5 ml to about 42 mg/5 ml. In another specific embodiment of the compositions and methods of the present invention, dextromethorphan tannate may be included in amounts ranging from about 28 mg/5 ml to about 48 mg/5 ml. In another specific embodiment of the compositions and methods of the present invention, dextromethorphan tannate may be included in an amount of about 38 mg/5 ml.

Phenylephrine ((S)-3-hydroxy-α [(methylamino)methyl]benzene-methanol) is a sympathomimetic amine. It acts as an oral nasal decongestant and laryngeal mucous membrane decongestant, with minimal central nervous stimulation, by stimulating alpha-adrenergic receptors to produce pronounced vasoconstriction in the skin, mucous membranes and the mucosa. In a specific embodiment, phenylephrine may be in the form of phenylephrine tannate. In a specific embodiment of the compositions and methods of the present invention, phenylephrine tannate may be included in amounts ranging from about 18 mg/5 ml to about 20 mg/5 ml. In another specific embodiment of the compositions and methods of the present invention, phenylephrine tannate may be included in amounts ranging from about 17 mg/5 ml to about 21 mg/5 ml. In another specific embodiment of the compositions and methods of the present invention, phenylephrine tannate may be included in amounts ranging from about 14 mg/5 ml to about 24 mg/5 ml. In another specific embodiment of the compositions and methods of the present invention, phenylephrine tannate may be included in an amount of about 19 mg/5 ml.

Viscous secretion exists in the airway of the human body. This secretion has an important role in imparting suitable temperature and humidity to inhaled air. When its amount is moderate, the secretion in the airway is unconsciously swallowed or expelled with the breath, but usually never is expectorated. Thus, any expectoration suggests that there is something extraordinary in the respiratory system. On the other hand, accumulation of this secretion in the airway is liable to cause an infection via the airway. From this point of view, the removal of the secretion is a matter of great significance in the medical treatment of patients who suffer with a disease in the airway.

In order to facilitate expectoration, medicines referred to as “expectorants” have been used. Most expectorants serve to remove the secretion by diluting it through an increase in secretion by the mucosa of the airway, promotion of separation from the mucosa and enhancement of ciliary beat. Guaifenesin (3-(2-methoxypphenoxy)-1,2-propanediol), also known as glyceryl guaiacolate, is an expectorant. It is readily absorbed from the intestinal tract and is thought to enter airway secretions unmetabolized and to have a direct effect either on the mucus secretion itself or the epithelium. Rubin, 116 CHEST 195-200 (1999). For example, guaifenesin is thought to reduce the thickness of mucus and phlegm secretions by increasing the production of fluids in the respiratory tract thus helping to liquefy and thin airway secretions. The increased flow of less viscid secretions promotes ciliary action and further facilitates the removal of airway secretions. Guaifenesin also may inhibit cough peripherally in the airway, by hydrating airway mucus so that it shields the cough receptors from cough-inducing irritants. Dicpinigaitis & Gayle, 124 CHEST 2178-2181 (2003). These peripheral actions of guaifenesin aid in the removal of accumulated secretions from the trachea, bronchi and lungs, thus changing a dry, non-productive cough to a cough that is more productive and less frequent. Guaifenesin also may act to suppress cough through an effect in the central nervous system. Rubin, supra. While the exact mechanism of this action of guaifenesin is not known, it is believed that guaifenesin acts centrally by depressing or blocking nerve impulse transmission at the internuncial neuron level of the subcortical areas of the brain, brainstem and spinal cord thus relaxing both the laryngeal and pharyngeal muscles.

In a specific embodiment of the compositions and methods of the present invention, guaifenesin may be included in amounts ranging from about 95 mg/5 ml to about 105 mg/5 ml. In another specific embodiment of the compositions and methods of the present invention, guaifenesin may be included in amounts ranging from about 90 mg/5 ml to about 110 mg/5 ml. In another specific embodiment of the compositions and methods of the present invention, guaifenesin may be included in amounts ranging from about 75 mg/5 ml to about 125 mg/5 ml. In another specific embodiment of the compositions and methods of the present invention, guaifenesin may be included in an amount of about 100 mg/5 ml.

In some embodiments of the present invention, the compositions may be substantially free of active ingredients other than guaifenesin, phenylephrine, and dextromethorphan. For example, in one embodiment, the compositions of the present invention may be substantially free of at least one other added antitussive. In another embodiment of the present invention, the compositions may be substantially free of at least one other added decongestant. In another embodiment of the present invention, the compositions may be substantially free of at least one other added nasal decongestant. In another embodiment of the present invention, the compositions may be substantially free of at least one other added opioid analgesic. In another embodiment of the present invention, the compositions may be substantially free of at least one other expectorant. In other embodiments of the present invention, the compositions may be substantially free of one or more other active ingredient, such as, but not limited to, antitussives, decongestants, nasal decongestants, opioid analgesics, and/or expectorants.

In other embodiments of the present invention, the compositions may additionally comprise one or more added active ingredients in addition to guaifenesin, phenylephrine, and dextromethorphan. For example, in one embodiment, the compositions of the present invention may comprise at least one other added antitussive. In another embodiment of the present invention, the compositions may comprise at least one other added decongestant. In another embodiment of the present invention, the compositions may comprise at least one other added nasal decongestant. In another embodiment of the present invention, the compositions may comprise at least one other opioid analgesic. In another embodiment of the present invention, the compositions may comprise at least one other expectorant. In other embodiments of the present invention, the compositions may comprise one or more other active ingredient, such as, but not limited to, antitussives, decongestants, nasal decongestants, opioid analgesics, and/or expectorants.

Antitussives of interest include, but are not limited to, codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan hydrobromide, chlophendianol hydrochloride, carbetapentane citrate, caramiphen edisylate, noscapine, noscapine hydrochloride, and menthol.

Decongestants of interest include, but are not limited to, ephedrine, ephedrine sulfate, ephedrine hydrochloride, pseudoephedrine hydrochloride, phenylephrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

Opioid analgesics of interest include, but are not limited to, such as, codeine, morphine, hydromorphone, hydrocodone, oxymorphone, levorphanol, fentanyl, propoxyphene, diphenoxylate, meperidine, methadone, oxycodone, butorphanol, benzonate and morphine.

Expectorants of interest include, but are not limited to ammonium chloride, ammonium carbonate, acetylcysteine, antimony potassium tartrate, glycerin, potassium iodide, sodium citrate, terpin hydrate, and tolu balsam.

Sugars found in various products are highly undesirable for a number of reasons. For instance, it is known that products, such as medications, containing a high sugar content, more particularly saccharose, fructose and dextrose, may attack the dental enamel as a result of acid being formed in the mouth by certain bacteria if the teeth are not cleaned properly. In addition, many sugar-containing products are unsuitable for diabetics because of their sugar content. Sugar found in manufactured products also may be undesirable due to the increased caloric content caused by the sugar. Because of these factors, there is a progressively increasing world demand for sugar free products, including medications.

Sugar free versions of products may be manufactured using sugar replacements, such as, for example and without limitation, saccharin sodium, maltodextrin, aspartame, potassium acesulfame, neohesperidin dihydrochalcone, sucralose, monoammonium glycyrrhizinate, and mixtures thereof. These “sugar replacements” have the advantage that they do not decompose to form products that attack the dental enamel as a result of the bacterial flora present in the mouth during metabolism, even if the teeth are not cleaned properly. The “sugar replacements” also are suitable for consumption by diabetics and do not add unneeded or unwanted calories to products such as medications. Thus, in a specific embodiment of the compositions and methods of the present invention, the compositions may be free of any added sugar, and instead, may contain a sugar substitute, such as for example and without limitation, one of the sugar replacements described above.

Many cough and decongestant medications also contain alcohol. For various legal, dietary, cultural and even religious reasons, there is interest in the production of alcohol-free medications. For instance, many alcohol-containing medications cannot be used by children because parents may be concerned about the alcohol content, and children may reject the alcohol bite and astringency characteristic of these products. Indeed, most alcohol-containing medications display the language “Keep Away From Children” on their labels. Similarly large numbers of adults reject alcohol based medications for various personal reasons. For instance and without limitation, those recovering from alcohol abuse avoid medications containing alcohol because of the threat that these substances can trigger a negative response. In addition, many institutionalized individuals are not allowed to use alcohol based medications. Therefore, there is a need for medications that are free of any added alcohol. In a specific embodiment of the compositions and methods of the present invention, the compositions may be free of any added alcohol.

The compositions and methods of the present invention may alleviate symptoms, such as coughing, sneezing, rhinorrhea, and/or nasal obstruction, caused by a variety of conditions. For instance, coughing, sneezing, rhinorrhea, and/or nasal obstruction may be caused by, for example and without limitation, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, or a respiratory disease, such as, for example and without limitation, a cold, acute bronchitis, chronic bronchitis, asthmatic bronchitis, bronchiectasis, pneumonia, lung tuberculosis, silicosis, silicotuberculosis, pulmonary cancer, upper respiratory inflammation (caused by, for example and without limitation, pharyngitis, laryngitis, nasal catarrh), asthma, bronchial asthma, infantile asthma, pulmonary emphysema, pneumoconiosis, pulmonary fibrosis, pulmonary silicosis, pulmonary suppuration, pleuritis, tonsillitis, cough hives, or whooping cough. The compositions and methods of the present invention may provide relief from symptoms caused by all of the above.

Typically cough and decongestant preparations are administered as an elixir in a sweetened aromatic solution of alcohol and water. In the present invention, however, syrups and other liquid vehicles also may be used. Although water itself may make up the entire carrier, typical cough formulations may contain a co-solvent, for example and without limitation, propylene glycol and/or glycerin, to assist solubilization and incorporation of water insoluble ingredients, flavorants and the like into the composition. Any such ingredients may be included as desired or needed within the compositions and methods of the present invention as long as they are consistent with the objectives herein defined. For example, it is contemplated that when desirable, flavoring, preserving, suspending, thickening and/or emulsifying agents may be included in the compositions and methods of the present invention. Formulations for orally administered mediciations are well known in the art. Descriptions of suitable formulations may be found in Remington, The Science and Practice of Pharmacy (A. Gennaro ed., 20^{th} ed., Lippincott, Williams & Wilkins, 2000).

Flavorants that may be used in accordance with the present invention include those known to those skilled in the art. These flavorants may include, for example and without limitation, natural, artificial and synthetic flavor oils and flavoring aromatic and/or oils, oleoresins and extracts derived from plants, animals, leaves, flowers, fruits, and so forth, and combinations thereof. Non-limiting representative flavor oils include anise oil, cinnamon oil, peppermint oil, spearmint oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil. Also useful flavorants include fruit essences including apple essence, pear essence, peach essence, berry essence, wildberry essence, date essence, blueberry essence, kiwi essence, strawberry essence, raspberry essence, cherry essence, plum essence, pineapple essence, and apricot essence. Other useful flavorants include aldehydes and esters such as benzaldehyde (cherry, almond), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), and 2-dodecenal (citrus, mandarin), mixtures thereof and the like. Honey and artificial honey flavor, as well as natural mixed berry flavor, citric acid, malic acid, vanilla, vanillin, cocoa, chocolate, and menthol may also be used in accordance with the present invention. Flavorants appealing to non-human patients may also be included in the composition of the invention, including but not limited to, yeast extract, meat extract, fish extract, poultry extract, cheese and other dairy flavors, and the like.

Another aspect of the invention is a method of administering to a patient the composition of the invention, comprising dextromethorphan tannate, phenylephrine tannate and guaifenesin. In some embodiments, the composition is administered to the patient orally. The composition of the invention may be administered in varying volumes and at varying frequencies. In specific embodiments, the dose volume is from about 1 to about 10 ml, or from about 0.1 to 100 ml. Specific dosages include, but are not limited to, 1.25 ml, 2.5 ml, 5.0 ml, and 10 ml. The frequency of the dose may vary from every other day to several times a day. In specific embodiments, the frequency of administration may be once a day, twice a day, three times a day or four times a day. In other specific embodiments, the frequency of the dose may be once a day or twice a day.

The dosage volume may depend in part with the characteristics of the patient. In some embodiments, the patient is a human over about 12 years of age and the composition may be administered in an about 5 ml to about 10 ml dose at least once a day or at least twice a day. In other embodiments, the patient is human from about 6 to about 12 years of age, and the composition of the invention is administered in an about 2.5 ml to about 5.0 ml dose once a day or twice a day. In another embodiment, the patient is a human from about 2 to about 6 years of age, and the composition of the invention is administered in an about 1.25 ml to about 2.5 ml dose once a day or twice a day.

The total dosage per day of the active compounds may be a factor in determining the criteria for administering the composition of the invention. For example, compositions with a higher concentration of active compounds may be taken in smaller dosages and/or less frequently, and compositions with lower concentrations of the active compounds may be taken in larger volume dosages and/or more frequently.

### EXAMPLES

Without further elaboration, it is believed that one skilled in the art, using the preceding description, can utilize the present invention to the fullest extent. The following examples are illustrative only, and not limiting of the remainder of the disclosure in any way whatsoever.

### Example 1

A composition of the following formulation is prepared in liquid swallowable form containing the following active ingredients per 5 ml teaspoonful:

| | |
|---|---|
| Guaifenesin | 100 mg |
| Phenylephrine tannate | 19 mg |
| Dextromethorphan tannate | 38 mg |

Other inactive ingredients include: citric acid, adetate disodium, glycerin, methylparaben, propylparaben, propylene glycol, saccharin sodium, sodium citrate, sorbitol, water, FD&C red 40, and artificial cherry flavor.

### Example 2

A study is undertaken to evaluate the effectiveness of the compositions of the present invention in the treatment of patients. The objective of the study is to determine whether oral intake of the compositions of the present invention results in an improvement of the symptoms of coughing, sneezing, rhinorrhea, and/or nasal obstruction.

A double-blind, placebo controlled study is conducted over a three-day period. A total of 120 subjects, all presenting for treatment of symptoms of coughing, sneezing, rhinorrhea, and/or nasal obstruction, are chosen for the study. The patients range in age from 8 to 72 years old.

An initial assessment of the symptoms of each patient is conducted when the patients initially present for treatment. The treating physician rates the severity of the symptoms on a 4-point scale (0:absent; 1:mild; 2: moderate; 3:severe). For inclusion in the study, a patient must be rated with a score of two or above for cough and a total score of at least 5 for the sum of the four selected symptoms.

The 120 subjects chosen for the study are separated into two separate groups of 60. The characteristics of the symptoms between the two groups are comparable. The first group is administered a 5 ml dose of the composition of the present invention every twelve hours for three days. The second group is administered a placebo medication every twelve hours for three days that is similar in all respects to the administered composition except for the exclusion of the active ingredients, dextromethorphan tannate, phenylephrine tannate and guaifenesin. No other medications are taken by the patients during the assessment period.

Patients self-evaluate their symptoms of coughing, sneezing, rhinorrhea, and nasal obstruction using the same 4-point scale (0:absent; 1:mild; 2:moderate; 3:severe) thirty minutes after each dose administration. Patients also note the presence and severity of adverse effects of taking the medication on the 4-point scale. In addition to the initial assessment on day 1, patients are evaluated at the end of day two and day three by the treating physician.

The data is evaluated using multiple linear regression analysis and a standard t-test. In each analysis, the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at each dose administration, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

This study will demonstrate the efficacy of the composition of the present invention in treating the symptoms of coughing, sneezing, rhinorrhea and nasal obstruction. Regarding potential adverse effects of taking the medication, if there are no significant differences between the two therapeutic groups, this study will demonstrate that the administration of the composition of the present invention is effective at treating symptoms of coughing, sneezing, rhinorrhea, and nasal obstruction, in addition to being well-tolerated by the patients.

While specific embodiments of the present invention have been described, other and further modifications and changes may be made without departing from the spirit of the invention. All further and other modifications and changes are included that come within the scope of the invention as set forth in the claims. The disclosures of all publications cited above are expressly incorporated by reference in their entireties to the same extent as if each were incorporated by reference individually.

What is claimed is:

1. A composition comprising dextromethorphan tannate, phenylephrine tannate and guaifenesin, wherein said composition is administrable to a patient.

2. The composition of claim 1, wherein said composition is substantially free of other added active ingredients.

3. The composition of claim 2, wherein said other added active ingredient is another antitussive.

4. The composition of claim 3, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

5. The composition of claim 2, wherein said other added active ingredient is another decongestant.

6. The composition of claim 5, wherein said another decongestant comprises a nasal decongestant.

7. The composition of claim 6, wherein said another nasal decongestant comprises one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

8. The composition of claim 2, wherein said other added active ingredient is another opioid analgesic.

9. The composition of claim 8, wherein said another opioid analgesic comprises one or more of the group consisting of codeine, morphine, hydromorphone, hydrocodone, oxymorphone, levorphanol, fentanyl, propoxyphene, diphenoxylate, meperidine, methadone, and oxycodone.

10. The composition of claim 2, wherein said other added active ingredient is another expectorant.

11. The composition of claim 10, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

12. The composition of claim 1, wherein said composition is substantially free of added sugar.

13. The composition of claim 1, wherein said composition is substantially free of added alcohol.

14. The composition of claim 1, wherein said composition is substantially free of added sugar and substantially free of added alcohol.

15. The composition of claim 1, wherein said composition is in a liquid form.

16. The composition of claim 1, wherein said composition further comprises a flavorant.

17. The composition of claim 16, wherein said flavorant comprises a natural flavorant.

18. The composition of claim 16, wherein said flavorant comprises an artificial flavorant.

19. The composition of claim 16, wherein said flavorant is selected from one or more of the group consisting of anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil.

20. The composition of claim 16, wherein said flavorant is selected from one or more of the group consisting of apple essence, pear essence, peach essence, berry essence, wildberry essence, date essence, blueberry essence, kiwi essence, strawberry essence, raspberry essence, cherry essence, plum essence, pineapple essence, and apricot essence.

21. The composition of claim 16, wherein said flavorant is selected from one or more of the group consisting of natural mixed berry flavor, citric acid, malic acid, vanilla, vanillin, cocoa, chocolate, and menthol.

22. The composition of claim 1, wherein said composition further comprises a non-sugar sweetening agent.

23. The composition of claim 22, wherein said non-sugar sweetening agent is selected from one or more of the group consisting of saccharin sodium, maltodextrin, aspartame, potassium acesulfame, neohesperidin dihydrochalcone, sucralose, and monoammonium glycyrrhizinate.

24. The composition of claim 1, wherein said composition further comprises citric acid, adetate disodium, glycerin, methylparaben, propylparaben, propylene glycol, saccharin sodium, sodium citrate, sorbitol, water, FD&C red 40, and artificial cherry flavor.

25. The composition of claim 1, wherein said composition comprises about 28 mg/5 ml to about 48 mg/5 ml dextromethorphan tannate.

26. The composition of claim 1, wherein said composition comprises about 14 mg/5 ml to about 24 mg/5 ml phenylephrine tannate.

27. The composition of claim 1, wherein said composition comprises about 75 mg/5 ml to about 125 mg/5 ml guaifenesin.

28. The composition of claim 1, wherein said composition comprises about 34 mg/5 ml to about 42 mg/5 ml dextromethorphan tannate.

29. The composition of claim 1, wherein said composition comprises about 17 mg/5 ml to about 21 mg/5 ml phenylephrine tannate.

30. The composition of claim 1, wherein said composition comprises about 90 mg/5 ml to about 110 mg/5 ml guaifenesin.

31. The composition of claim 1, wherein said composition comprises about 36 mg/5 ml to about 40 mg/5 ml dextromethorphan tannate.

32. The composition of claim 1, wherein said composition comprises about 18 mg/5 ml to about 20 mg/5 ml phenylephrine tannate.

33. The composition of claim 1, wherein said composition comprises about 95 mg/5 ml to about 105 mg/5 ml guaifenesin.

34. The composition of claim 1, wherein said composition comprises about 36 mg/5 ml to about 40 mg/5 ml dextromethorphan tannate; about 18 mg/5 ml to about 20 mg/5 ml phenylephrine tannate; and about 95 mg/5 ml to about 105 mg/5 ml guaifenesin.

35. The composition of claim 1, wherein said composition comprises about 3.8 mg/5 ml dextromethorphan tannate.

36. The composition of claim 1, wherein said composition comprises about 19 mg/5 ml phenylephrine tannate.

37. The composition of claim 1, wherein said composition comprises about 100 mg/5 ml guaifenesin.

38. The composition of claim 1, wherein said composition comprises about 38 mg/5 ml dextromethorphan tannate; about 19 mg/5 ml phenylephrine tannate; and about 100 mg/5 ml guaifenesin.

39. A method comprising administering to a patient a composition comprising the composition of claim 1.

40. The method of claim 39, wherein the composition is administered to the patient orally.

41. The method of claim 39 wherein the composition is administered to the patient at a frequency selected from the group consisting of once a day, twice a day, three times a day and four times a day.

42. The method of claim 39, wherein the composition is administered to the patient in a dose from about 0.1 ml to about 100 ml.

43. The method of claim 39, wherein the patient is suffering from at least one condition selected from the group consisting of coughing, sneezing, rhinorrhea, nasal obstruction, nasal congestion, nasal pruritus, rhinorrhea, allergies, allergic vasomotor rhinitis (hay fever), seasonal allergic vasomotor rhinitis, perennial allergic vasomotor rhinitis, bronchography, bronchoscopy, a respiratory disease, a cold, acute bronchitis, chronic bronchitis, asthmatic bronchitis, bronchiectasis, pneumonia, lung tuberculosis, silicosis, silicotuberculosis, pulmonary cancer, upper respiratory inflammation, pharyngitis, laryngitis, nasal catarrh, asthma, bronchial asthma, infantile asthma, pulmonary emphysema, pneumoconiosis, pulmonary fibrosis, pulmonary silicosis, pulmonary suppuration, pleuritis, tonsillitis, cough hives, and whooping cough.

44. The method of claim 39, wherein said composition is substantially free of other added active ingredients.

45. The method of claim 44, wherein said other added active ingredient is another antitussive.

46. The method of claim 45, wherein said another antitussive comprises one or more of the group consisting of codeine, codeine phosphate, codeine sulfate, morphine, morphine sulfate, hydromorphone hydrochloride, levorphanol tartrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, dextromethorphan, dextromethorphan hydrobromide, chlophendianol hydrochloride, carbetapentane citrate, caramiphen edisylate, noscapine, noscapine hydrochloride, and menthol.

47. The method of claim 44, wherein said other added active ingredient is another decongestant.

48. The method of claim 47, wherein said another decongestant comprises a nasal decongestant.

49. The method of claim 48, wherein said another nasal decongestant comprises one or more of the group consisting of ephedrine, ephedrine sulfate, ephedrine hydrochloride, psuedoephedrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride, and xylometazoline hydrochloride.

50. The method of claim 44, wherein said other added active ingredient is another opioid analgesic.

51. The method of claim 50, wherein said another opioid analgesic comprises one or more of the group consisting of codeine, morphine, hydromorphone, hydrocodone, oxymorphone, levorphanol, fentanyl, propoxyphene, diphenoxylate, meperidine, methadone, and oxycodone.

52. The method of claim 44, wherein said other added active ingredient is another expectorant.

53. The method of claim 52, wherein said another expectorant comprises one or more of the group consisting of ammonium chloride, ammonium carbonate, acetylcysteine, antimony potassium tartrate, glycerin, potassium iodide, sodium citrate, terpin hydrate, and tolu balsam.

54. The method of claim 39, wherein said composition is substantially free of added sugar.

55. The method of claim 39, wherein said composition is substantially free of added alcohol.

56. The method of claim 39, wherein said composition is substantially free of added sugar and substantially free of added alcohol.

57. The method of claim 39, wherein said composition is in a liquid form.

58. The method of claim 39, wherein said composition further comprises a flavorant.

59. The method of claim 58, wherein said flavorant comprises a natural flavorant.

60. The method of claim 58, wherein said flavorant comprises an artificial flavorant.

61. The method of claim 58, wherein said flavorant is selected from one or more of the group consisting of anise oil, cinnamon oil, peppermint oil, spearmint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, and grape oil.

62. The method of claim 58, wherein said flavorant is selected from one or more of the group consisting of apple essence, pear essence, peach essence, berry essence, wildberry essence, date essence, blueberry essence, kiwi essence, strawberry essence, raspberry essence, cherry essence, plum essence, pineapple essence, and apricot essence.

63. The method of claim 58, wherein said flavorant is selected from one or more of the group consisting of natural mixed berry flavor, citric acid, malic acid, vanilla, vanillin, cocoa, chocolate, and menthol.

64. The method of claim 39, wherein said composition further comprises a non-sugar sweetening agent.

65. The method of claim 64, wherein said non-sugar sweetening agent is selected from one or more of the group consisting of saccharin sodium, maltodextrin, aspartame, potassium acesulfame, neohesperidin dihydrochalcone, sucralose, and monoammonium glycyrrhizinate.

66. The method of claim 39, wherein said composition further comprises citric acid, adetate disodium, glycerin, methylparaben, propylparaben, propylene glycol, saccharin sodium, sodium citrate, sorbitol, water, FD&C red 40, and artificial cherry flavor.

67. The method of claim 39, wherein said composition comprises about 28 mg/5 ml to about 48 mg/5 ml dextromethorphan tannate.

68. The method of claim 39, wherein said composition comprises about 14 mg/5 ml to about 24 mg/5 ml phenylephrine tannate.

69. The method of claim 39, wherein said composition comprises about 75 mg/5 ml to about 125 mg/5 ml guaifenesin.

70. The composition of claim 39, wherein said composition comprises about 34 mg/5 ml to about 47 mg/5 ml dextromethorphan tannate.

71. The method of claim 39, wherein said composition comprises about 17 mg/5 ml to about 24 mg/5 ml phenylephrine tannate.

72. The method of claim 39, wherein said composition comprises about 90 mg/5 ml to about 110 mg/5 ml guaifenesin.

73. The method of claim 39, wherein said composition comprises about 36 mg/5 ml to about 40 mg/5 ml dextromethorphan tannate.

74. The method of claim 39, wherein said composition comprises about 18 mg/5 ml to about 20 mg/5 ml phenylephrine tannate.

75. The method of claim 39, wherein said composition comprises about 95 mg/5 ml to about 105 mg/5 ml guaifenesin.

76. The method of claim 39, wherein said composition comprises about 36 mg/5 ml to about 40 mg/5 ml dextromethorphan tannate; about 18 mg/5 ml to about 20 mg/5 ml phenylephrine tannate; and about 95 mg/5 ml to about 105 mg/5 ml guaifenesin.

77. The method of claim 39, wherein said composition comprises about 38 mg/5 ml dextromethorphan tannate.

78. The method of claim 39, wherein said composition comprises about 19 mg/5 ml phenylephrine tannate.

79. The method of claim 39, wherein said composition comprises about 100 mg/5 ml guaifenesin.

80. The method of claim 39, wherein said composition comprises about 38 mg/5 ml dextromethorphan tannate; about 19 mg/5 ml phenylephrine tannate; and about 100 mg/5 ml guaifenesin.
